(19) United States
(12) Patent Application Publication
(10) Pub. No.: US 2002/0128611 A1
(43) Pub. Date: Sep. 12, 2002

(54) Fluid delivery apparatus

(76) Inventor: Naji Kandalaft, Mission, TX (US)

Correspondence Address: Naji Kandalaft, M.D, 1008 Rio Grande Drive, Mission, TX, 78572, US

(21) Appl. No.: 09/727,148

(22) Filed: Dec. 1, 2000

(51) Int. Cl. A61M 5/00

(52) U.S. Cl. 604/246; 251/7

(57) ABSTRACT

Improved fluid delivery apparatus for use in a variety of medical catheterization procedures, such as angiography are disclosed. In one aspect, the apparatus includes a manifold having a lumen and a first port connected to the lumen. The first port has a first branch for connecting to a first fluid reservoir and a second branch for connecting to a second fluid reservoir which is usually radiopaque material (dye fluid). The apparatus also includes a shutter assembly that facilitates control of fluid flow from the first fluid reservoir, from the second fluid reservoir, and through the lumen. In another aspect, the fluid delivery apparatus includes a manifold having a lumen with a first end for connecting to a catheter and a second end for connecting to a syringe. The manifold also has a first port proximate the catheter with a first end connected to the lumen and a second end for connecting to a pressure measuring means. The apparatus also includes a shutter assembly that facilitates generally constant, automatic measurements of a patient's blood pressure, without the need to repeatedly move external controls.

### FIELD OF THE INVENTION

The present invention pertains to fluid delivery apparatus, and more particularly but not by way of limitation, to an improved fluid delivery apparatus used in a variety of medical catheterization procedures.

### HISTORY OF THE RELATED ART

In medical catheterization procedures such as angiography avoiding the accidental injection of air emboli into a patient's r vascular system is of great importance. The injection of an air bubble into a vein or artery while administering fluid can result in severe medical complications or even death.

Conventional fluid delivery apparatus often aspirate fluid from a reservoir by creating a vacuum. In such apparatus risk for accidental injection of air into a patient exists as fluid is drawn from the reservoir by a vacuum for subsequent injection into a patient, gas from suspension in the fluid may also be drawn. These drawn gases may be introduced into the vascular system of the patient upon injection of the fluid.

Conventional fluid delivery apparatus are often pressurized to aid in avoiding such accidental injection of air. In such pressurized systems fluid is forced under pressure into the syringe of the fluid delivery apparatus and the potential for drawing gas from suspension is greatly reduced or eliminated.

In angiography conventional fluid delivery apparatus are used to deliver saline and radiopaque substance. A catheter is inserted into a patient's artery and a radiopaque substance is injected into the artery by the fluid delivery apparatus and an x-ray is taken of the artery or particular organ of interest.

Different sizes and shapes of catheters are often required to access particular arteries or organs and therefore multiple catheters may be required for a particular angiographic procedure. Each time a new catheter is required the old catheter must be disconnected from the fluid delivery apparatus, the new catheter must be connected to the fluid delivery apparatus and the new catheter must be aspirated with fluid and cleared of all air bubbles.

Each of these steps must be performed before the fluid delivery apparatus may be used to inject radiopaque substance into the patient. Conventional fluid delivery apparatus for radiopaque substances may be manually operated or electronically (and often a microprocessor) controlled system. The present invention is particularly directed to a hand-held manual system.

FIG. 1 shows an exemplary conventional, manually operated fluid delivery apparatus 10, which is sold under the name of Morse Manifold or (three way manual manifold) by NAMIC, corporation of Schneider. Fluid delivery apparatus 10 generally includes a manifold 12 having an axial main lumen 14. Lumen 14 is connected on a first end to a catheter connector 16 and on a second end to a syringe connector 18. Catheter connector 16 and syringe connector 18 allow connections to be formed to a catheter 19 and to a syringe 20 respectively using mating, quick release threads (not shown).

Apparatus 10 further includes three-way stopcocks 22, 24, and 26. Stopcock 22 comprises a port 28, a shutter 30 located within lumen 14 and an exterior handle 32 for actuating shutter 30. Port 28 is connected to a pressure transducer pressure gauge or other conventional pressure measuring means 33 by conventional flexible tubing (not shown). Handle 32 may be turned to actuate shutter 30 to three general positions. The first position places port 28 in fluid communication with lumen 14 and to syringe connector 18. The second position allows fluid flow to lumen 14 on both sides of stopcock 22 but does not allow fluid flow to port 28. The third position open port 28 to catheter connector 16. Stopcock 24 comprises a port 34 shutter 36 located within lumen 14 and an exterior handle 38 for actuating shutter 36.

Port 34 is connected to a saline reservoir 37 by conventional flexible tubing (not shown). Saline reservoir 37 is typically pressurized using a pressure infuser bag surrounding reservoir 37 or other conventional pressurization means (not shown). Handle 38 may be turned to actuate shutter 36 to three general positions. The first position places port 34 in fluid communication with syringe connector 18. The second position allows fluid flow through lumen 14 on both sides of stopcock 24 but does not allow fluid flow to port 34. The third position open port 34 to catheter connector 16.

Stopcock 26 comprises a port 40, a shutter 42 located within lumen 14, and an exterior handle 44 for actuating shutter 42. Port 40 is connected to a radiopaque substance reservoir 45 by conventional flexible tubing (not shown), handle 44 may be turned to actuate shutter 42 to three general positions. The first position places port 40 in fluid communication with the syringe connector 18; the second position allows fluid to flow toward lumen 14 on both sides of stopcock 26 but does not allow fluid flow to port 40. The third position open port 40 to catheter connector 16.

During angiography a doctor first performs the following procedure to initiate fluid delivery apparatus 10: first be doctor turn stopcock 26 to position one make fluid connection between radiopaque substance and syringe connector 18, second he turns stopcock 22 to position 1 and stopcock 24 to position three making fluid connection between pressurized reservoir 37 and pressure measurement apparatus 33. Third he turns stopcock 22, 24, and 26 to position two making fluid connections through lumen 14.

The doctor then inserts catheter 19 into the desired area of the vascular system of a patient then flushes the catheter free of any air bubbles and connect it to the manifold 10. Before a doctor may begin the injection of a radiopaque substance he or she must clear air present in catheter 19 and in lumen 14 using the following procedure:

First, the doctor inverts apparatus 10 so that the catheter connector 16 is pointing generally downward toward the ground. Second, the doctor taps gently on manifold 12 to free any air bubbles that might be adhering to the inside surfaces of catheter 19 or lumen 14. Third, the doctor moves a plunger 46 of syringe 20 from position A to position B as shown in FIG. 1, drawing any air bubbles in catheter 19 and lumen 14 to be back off syringe 20 proximate to position B. Fourth, having cleared catheter 19 and lumen 14 of all air bubbles and while still holding apparatus 10 in an inverted position, the doctor then moves plunger 46 from position B to position A injecting a portion of the blood and saline in lumen 14 but not any air bubbles present in syringe 20 through catheter 19 into the patient's vascular system. In the fifth step, the doctor has to turn stopcock 28 to third position for pressure measurement.

To inject a radiopaque substance such as a contrast dye from apparatus 10, a doctor follows the following procedure: first the doctor turns stopcock 26 to first position to make connection with syringe 18, second the doctor moves plunger 46 from position A to position B, to aspirate radiopaque substance into the syringe, third he turns stopcock 26 to position two making connection to lumen 14, fourth he turns stopcock 22 and 24 to position two making connection between syringe 18 and catheter 19, fifth the doctor moves plunger 46 from position B to position A to inject dye into the catheter and into the patient's vascular system. Typically the doctor has 3 to 5 second time period in which to take x-rays as a dye flows through the artery or organ of interest. After taking an x-ray the doctor has to turn stopcock 22 to its 3d position so that the patient's blood pressure reading may be taken via pressure transducer 33, the doctor typically takes between two to five x-rays for each artery or organ of interest and for each x-ray the above described injection procedure must be repeated, in addition some organs require multiple catheters to get a complete x-ray of the organ. For example three different catheters having different sizes and shapes are typically used during angiography of the heart, of course each time a new catheter must be utilized the above described air cleaning procedures must be repeated.

During angiography it is also necessary to prevent blood from sitting motionless in catheter 19 to reduce the risk of clots forming and being introduced into the patient's vascular system, therefore a doctor may periodically flush catheter 19 using the following procedure: first the doctor sets stopcock 24 to its first position placing reservoir 37 in fluid communication with lumen 14 and syringe 20 via port 34, second the doctor then moves plunger 46 from position A to position B drawing saline from reservoir 37 into syringe 20, third the doctor sets stopcock 22, 24, and 26 to their second positions opening lumen 14 from catheter 19 to syringe 20 but closing port 28, 34, and 40, fourth the doctor then moves plunger 46 from position B to position A while holding apparatus 10 in inverted position injecting saline but not any air bubbles present in syringe 20 through lumen 14 and catheter 19 into the patient's vascular system.

While conventional, manually operated fluid delivery apparatus 10 has proved to be generally effective for angiography, certain limitations still remain. As described above the initialization procedure, the injection procedure and the flushing procedure for apparatus 10 each require several steps. In each of these procedures, some of the steps involve turning the handles of the stopcocks. This constant manipulation of the stopcocks requires a doctor to use two hands to operate apparatus 10 and complicate the angiographic procedure. In addition a doctor also has to manipulate a stopcock whenever he wants to measure the patient's blood pressure. Therefore a need exists in the medical industry for an improved manually operated fluid delivery apparatus that requires minimal manipulation of stopcocks or similar controls, that can generally be operated with one hand, that provides constant blood pressure measurements and that simplifies the angiographic procedure while safely delivering fluids to the vascular system of the patient.

### SUMMARY OF THE INVENTION

The present invention pertains to improved fluid delivery apparatus. More particularly, one aspect of the present invention comprises a fluid delivery apparatus including a manifold having a lumen and a first port connected to the lumen. The first port has a first branch for connecting to a reservoir for radiopaque substance and a second branch for connecting to a fluid reservoir. The apparatus also includes a shutter having first and second transverse bores that is slidably disposed within the first and second branches. The shutter is moveable to a first position in which the first transverse bore places the first reservoir of radiopaque substance (the dye fluid) in fluid communication with the first port, and the shutter is moveable to a second position in which the second transverse bore places the fluid reservoir (flush fluid) in fluid communication with the first port. When such an apparatus is used in a medical catheterization procedure, such as an angiography, it simplifies the initialization procedure, the injection procedure, and the flushing procedure relative to conventional fluid delivery apparatus.

In another aspect, the present invention comprises a fluid delivery apparatus for delivering fluid to the vascular system of a patient. The apparatus includes a manifold having a lumen with a first end for connecting to a catheter and a second end for connecting to a syringe. The manifold also has a first port proximate the catheter with a first end connected to the lumen and a second end for connecting to a pressure measuring means. The apparatus also includes a first valve rotatably coupled to an anterior surface of the lumen proximate to the first port and a spring coupled to the first valve biasing the first valve against rotation in a direction towards the catheter. When such an apparatus is used in a medical catheterization procedure, such as angiography, it provides generally constant automatic measurements of the patient's blood pressure without the need to repeatedly move external controls.

### BRIEF DESCRIPTION OF THE DRAWINGS

For a more complete understanding of the present invention and for further objects and advantages thereof, reference may now be had to the following description taken in conjunction with the company drawings in which:

FIG. 1 is a cross sectional schematic view of a conventional manually operated fluid delivery apparatus.

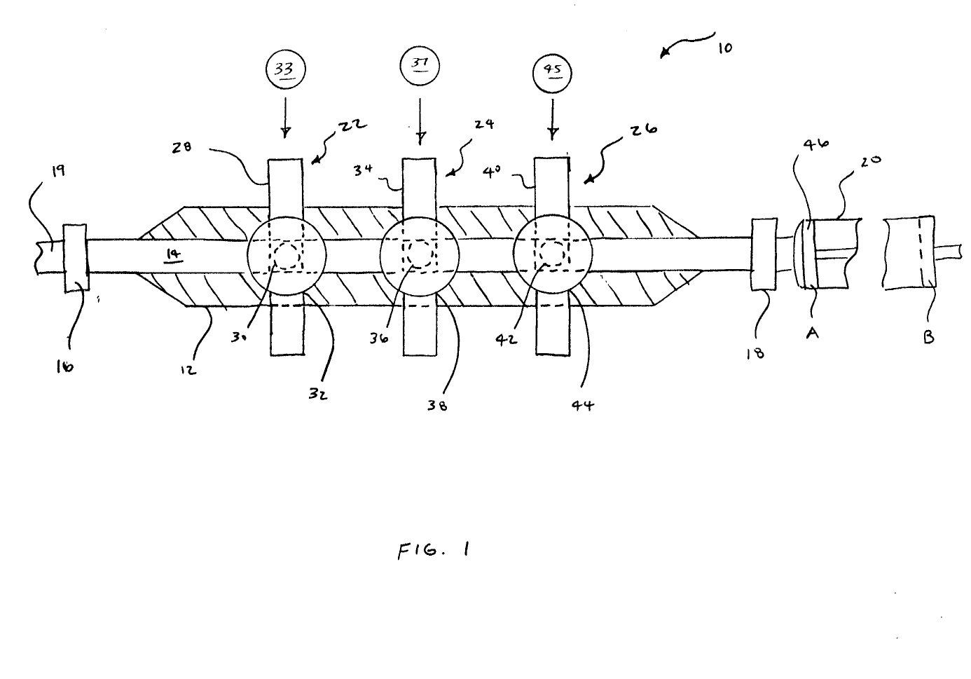

FIG. 2 is a cross sectional schematic view of a fluid delivery apparatus according to a first referred embodiment of the present invention.

FIG. 3 is a cross sectional schematic view of a fluid delivery apparatus according to a second preferred embodiment of the present invention.

### DETAILED DESCRIPTION OF THE PREFERRED EMBODIMENTS

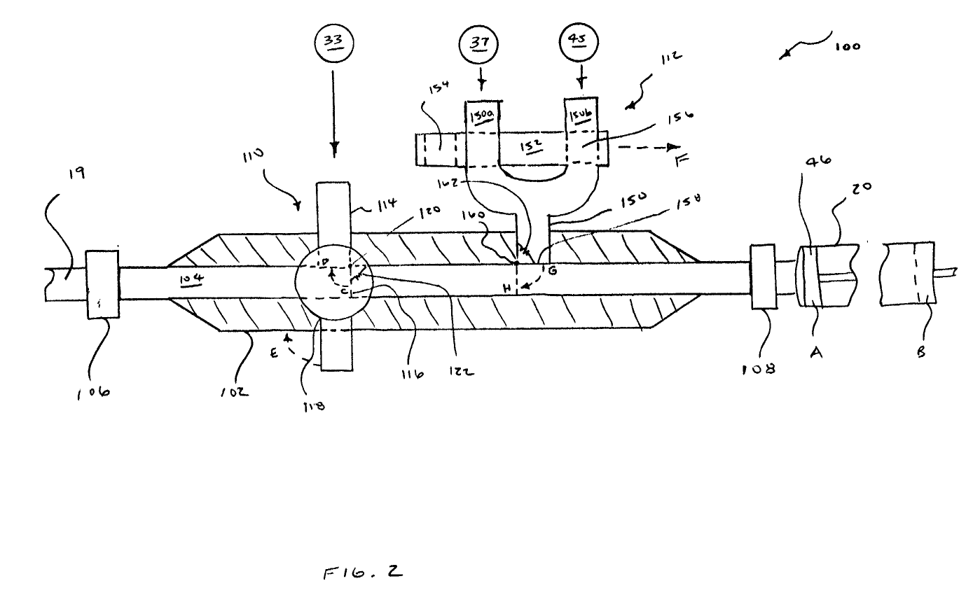

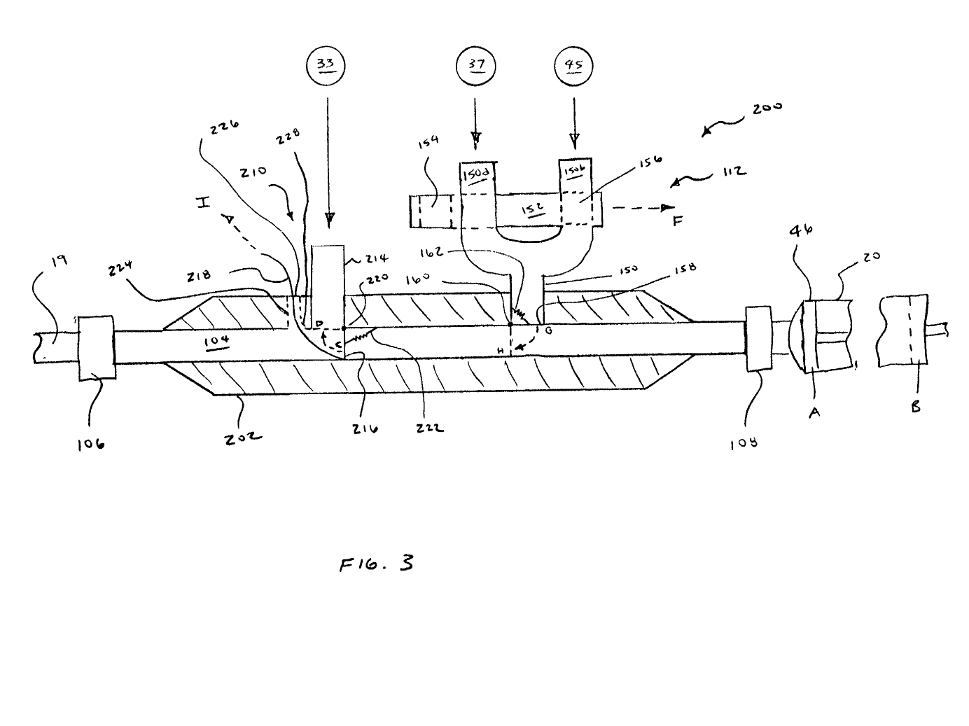

The preferred embodiments of the present invention and their advantages are best understood by referring to FIGS. 2-3 of the drawings, like numerals being used for like and corresponding parts of the various drawings.

Referring to FIG. 2, a cross sectional view of a fluid delivery apparatus according to a first preferred embodiment of the present invention is shown. Fluid delivery apparatus 100 comprises a manifold 102 having an axial main lumen 104. Lumen 104 is connected on a first end to a catheter connector 106 and on a second end to a syringe connector 108. Catheter connector 106 and syringe connector 108 allow connections to be formed to catheter 19 and to syringe 20 respectively using mating quick release threads (not shown).

Apparatus 100 further comprises shutter assemblies 110 and 112. Shutter assembly 110 comprises a port 114, a valve 116 located within lumen 104 and an exterior handle 118 for actuating valve 116. Valve 116 is rotatably coupled to an anterior surface of lumen 104 via hinge 120. Hinge 120 preferably allows valve 116 to rotate only from a position C to position D in lumen 104 as shown in FIG. 2. Therefore hinge 120 preferably prevents valve 116 from rotating a clockwise direction past position D, and hinge 120 preferably prevents valve 116 from rotating in a counterclockwise direction past position C. Alternatively, although not shown in FIG. 2 a stop or other abutment may extend from a left interior surface of port 114 and from a lower interior surface of lumen 104 to limit the rotation of valve 116 around hinge 120 as described above. Valve 116 is biased against clockwise movement by a spring 122. Although spring 122 is shown in FIG. 2 as a coil spring coupled on one end to valve 116 and to the upper interior surface of lumen 104 on an opposite end, other conventional springs may be employed with valve 116. Port 114 is connected to a pressure transducer, a pressure gauge, or other conventional pressure measuring means 33 by conventional flexible tubing (not shown). Handle 118 is rotatably coupled to hinge 120. Therefore, handle 118 may be used to actuate valve 116 to two general positions. As shown in FIG. 2 handle 118 is actuated so that valve 116 is in position C, its natural or resting position. Position C allows fluid communications between port 114 and lumen 104 but does not allow fluid flow through lumen 104 past valve 116. If handle 118 is turned 90° clockwise from the position shown in FIG. 2 as indicated by arrow E, the force of spring 122 is overcome and valve 116 is moved from position C to position D. Position D allows fluid flow through lumen 104 but does not allow fluid flow to port 114.

Shutter assembly 112 comprises a port 150 and a shutter 152. Port 150 has two branches, a branch 150a and a branch 150b. Branch 150a is connected to saline reservoir 37 via conventional flexible tubing (not shown). Saline reservoir 37 is preferably is pressurized using a pressure infuser bag surrounding reservoir 37 or other conventional pressurization means (not shown). Branch 150b is connected to radiopaque substance reservoir 45 via conventional flexible tubing (not shown).

Shutter 152 is slidably coupled within branches 150a and 150b, and shutter 152 has two transverse bores 154 and 156. Shutter 152 may be actuated to two general positions. As shown in FIG. 2, shutter 152 is in a first position in which transverse bore 156 is aligned with branch 150b. This first position allows fluid communications between radiopaque substance reservoir 45 and port 150, but prevents fluid communication between saline reservoir 37 and port 150. If shutter 152 is pushed in a direction of arrow F in FIG. 2, it may be actuated to a second position in which transverse bore 154 is aligned with branch 150a. The second position allows fluid communication between saline reservoir 37 and port 150, but prevents fluid communication between raqiopaque substance reservoir 44 and port 150.

Shutter assembly 112 further comprises a valve 158. Valve 158 is rotatably coupled to an interior surface of lumen 104 via hinge 160. Hinge 160 preferably allows valve 158 to rotate only from a position G to a position H in lumen 104 as shown in FIG. 2. Therefore hinge 160 preferably prevents valve 158 from rotating in a clockwise direction past position H and hinge 160 preferably prevents valve 158 from rotating in a counterclockwise direction past position G. Alternatively although not shown in FIG. 2, a stop or other abutment may extend from a right interior surface of port 150 and from a lower anterior surface of lumen 104 to limit the rotation of valve 158 around hinge 160 as described above. Valve 158 is biased against clockwise movement by a spring 162. Although spring 162 is shown in FIG. 2 as a coil spring coupled on one end to valve 158 and to the left anterior surface of port 150 on an opposite end, other conventional springs may be employed with valve 158.

When valve 158 is in its natural or resting position G, fluid may flow through lumen 104 past valve 158, but fluid may not flow into port 150. When valve 158 is in position H, port 150 is in fluid communication with lumen 104 but fluid may not flow past valve 158.

During angiography, a doctor may initialize fluid delivery apparatus 100. First, the doctor inserts catheter 19 into the desired area of the vascular system of the patient. Second, the doctor connects catheter 19 to manifold 102 via catheter connector 106.

Before beginning the injection of a radiopaque substance, the doctor must clear air present in catheter 19 and in lumen 104 using the following procedure. While handle 118 is down and valve 116 in position C, first the doctor moves shutter 152 to its second position placing reservoir 37 in fluid communication with port 150. At this point, saline does not flow freely from port 150 into lumen 104 because the flow of saline does not overcome the force of spring 162. If reservoir 37 is pressurized, the pressure is preferably selected so as to prevent the drawing of gas from suspension in the saline and so that the pressurized saline does not overcome the force of spring 162. Second, the doctor aspirates with negative pressure on syringe 20 moving the plunger 46 from position A to position B aspirating normal saline into the syringe. Third, the doctor moves handle 118 horizontally and tracing valve 116 to position D clockwise that will place the lumen of catheter 19 and lumen 104 in free fluid connections. Fourth, the doctor inverts the manifold downward pointing towards catheter 19 and tapping gently on manifold 102 to free any air bubbles that might be adhering to the inside surface of catheter 19 and lumen 104. While applying a gentle negative pressure on plunger 46 and moving plunger 46 from position A to position B, the doctor aspirates mixed blood and saline in lumen 104 to syringe 20 and also removing all air bubbles from catheter 19 and lumen 104 to back of syringe 20 proximate position B. While still holding apparatus 10 in an inverted position, the doctor then moves plunger 46 from position B to position A injecting a portion of the blood and saline into lumen 104 but not any air bubbles present in syringe 20 through catheter 19 into the patient's vascular system. Now the system is ready to be operable. Next, the doctor has to move handle 118 back to its vertical position and by that moving valve 116 counterclockwise back to its resting position C that puts lumen 104 and catheter 19 in connection with pressure measurement port 33. Next, the doctor has to move shutter 152 to its first position placing reservoir 45 that has the dye fluid (radiopaque substance) in connection with port 150.

To operate the system and to inject radiopaque substance into the patient's system, the doctor can do that with only two easy steps. First, by moving plunger 46 from position A to position B we create a vacuum in lumen 104 in the area generally below valve 158. This vacuum allows the contrast dye in port 150 to overcome the force of spring 162 automatically moving valve 158 from position G to position H and drawing contrast dye from port 152 into lumen 104 and into syringe 20. When the pressure in port 150 and lumen 104 equalize, spring 162 moves valve 158 back to position G, its resting position. Second, the doctor pushes plunger 46 from position B to position A creating pressurized area in lumen 104 to overcome the force of spring 122 and moves valve 116 clockwise from position C to position D allowing free flow of radiopaque substance from syringe 20 to lumen 104 to catheter 19 to the patient's vascular system. Releasing pressure of plunger 46 automatically allows valve 116 back to its resting position C. In addition, the patient's pressure is transmitted through catheter 19 into lumen 104 to force spring 122 and moves valve 116 back to its resting position C. This automatic movement allows instant and intermediate pressure measurement of the patient's blood pressure by putting catheter 19 and lumen 104 in connection with pressure gauge 33. The doctor is ready immediately for the next injection by repeating the first and second steps of moving the plunger 46 from position A to position B and back from position B to position A. This movement allows valve 158 to move from position G to position H during first plunger movement from A to B and force valve 116 to move from position C to position D during second movement of plunger from position B to position A during injection of dye into catheter 19 and into the vascular system of the patient.

As the contrast dye enters the patient's vascular system, the doctor typically has three to five seconds time in which to take x-rays as the dye flows through the artery or organ of interest. The doctor typically takes between two to five x-ray for each artery or organ of interest and for each x-ray the simple movement of plunger 46 from position A to B and back to position A is sufficient for dye infection into the artery or organ of interest. These two steps is all required for the new and improved fluid delivery apparatus 100 compared to the old system that is presently in use. The injection procedure of apparatus 100 is a significant improvement over that of conventional fluid delivery apparatus 10. In addition, unlike conventional fluid delivery apparatus 10, fluid delivery apparatus 100 provides the advantage of generally constant automatic blood pressure measurements without the need for repeatedly moving external controls. As noted previously, some organs require multiple catheters to get a complete x-ray of the organ and each time a new catheter must be utilized, the above described initialization and air clearing procedures must also be repeated for apparatus 100. However, the initialization procedure of apparatus 100 which include air cleaning procedure, requires only three steps, only two of them require the movement of an exterior handle. Therefore the initialization procedure of apparatus 100 is also improved from that of conventional apparatus 10.

Referring now to FIG. 3, a cross sectional view of a fluid delivery apparatus according a second preferred embodiment of the present invention is shown. Fluid delivery apparatus 200 has a structure substantially identical to that of fluid delivery apparatus 100, with the following exceptions. First, shutter assembly 110 of apparatus 100 is replaced with shutter assembly 210 in apparatus 200. Second, apparatus 200 has a manifold 202 that is slightly modified from manifold 102 of apparatus 100 as is described in greater detail hereinbelow. Shutter assembly 210 comprises port 214, valve 216 located within lumen 104 and a string 218 for actuating valve 216. Valve 216 is rotatably coupled to an anterior surface of lumen 104 via hinge 220. Hinge 220 preferably allows valve 216 to rotate only from a position C to position D in lumen 104 as shown in FIG. 3. Therefore hinge 220 preferably prevents valve 216 from rotating a clockwise direction past position D and hinge 220 preferably prevents valve 216 from rotating in a counterclockwise direction past position C. Alternatively, although not shown in FIG. 3 a stop or other abutment may extend from a left, interior surface of port 214 and from a lower interior surface of lumen 104 to limit the rotation of valve 216 around hinge 220 as described above. Valve 216 is biased against clockwise movement by a spring 222. Although spring 222 is shown in FIG. 3 as a coil spring coupled on one end to valve 216 and to the upper interior surface of lumen 104 on an opposite end, other conventional springs may be employed with valve 216. Port 214 is connected to a pressure transducer 33 by conventional flexible tubing (not shown). Manifold 202 is modified from manifold 102 of apparatus 100 in the following manner: Port 224 is found in manifold 202, proximate port 214. The lower end of port 224 is open to lumen 104, and the upper end of port 224 contains an annular seal 226.

String 218 has a first end coupled to valve 216 and a second end position on the exterior of apparatus 200. String 218 passes through port 224 and the aperture of annular seal 226. The aperture of annular seal 226 and string 218 are sized so as to create a fluid tight seal between the interior of port 224 and the exterior of apparatus 200, while allowing string 218 to be sidably removed through the aperture of annular seal 226. Port 224 preferably has a rounded interior edge 228 that minimizes friction during movement of string 218.

String 218 may be used to actuate valve 216 to two general positions. As shown in FIG. 3, valve 216 is in position C, its natural resting position. Position C allows fluid communications between port 214 and lumen 104, but does not allow fluid flow through lumen 104 past valve 216. If string 218 is pulled in a generally upward direction as shown by arrow I in FIG. 3, the force of spring 222 is overcome and valve 216 is moved from position C to position D. The position D allows fluid flow through lumen 104 but does not allow fluid flow through port 214. Port 214 is usually connected to pressure measuring means.

During angiography, the doctor may initialize fluid delivery apparatus 200 using only three steps. First, the doctor inserts catheter 19 into the desired area of the vascular system of the patient. Second, the doctor connects catheter 19 to manifold 202 via catheter connector 106. Third, the doctor moves shutter 152 to a second position (in direction of arrow F) allowing communications of reservoir 37 that usually has saline solution into port 150. By moving plunger 46 from position A to position B, overcoming the force of the spring 162, the doctor aspirates saline solution into syringe 20. Third, by pulling string 218 in direction of arrow I and holding string 218 to set valve 216 to position D opening lumen 104 from catheter 19 to syringe 20 closing port 214. Tapping gently on manifold 202 while in inverted position, the doctor can aspirate any air bubbles that might be adhered to the anterior surface of catheter 19 and lumen 104 all the way back to syringe 20. One should note that the doctor may easily hold string 218 between one of his or her fingers and the exterior surface of port 214. By releasing string 218 the doctor allows valve 216 to go back vertically to its resting position C and put lumen 104 and direct communications with port 214 to pressure gauge allowing direct and intermediate blood pressure measurement of the patient. Finally, the doctor moves shutter 152 back into its first position allowing communications from reservoir 45 which has a radiopaque substance into port 150. Now the system is ready and is operable. To take pictures of the patient's vascular system, the doctor can accomplish that by two easy steps: 1) by moving the plunger 46 from position A to position B creating a vacuum and lumen 104 under valve 158 and allowing radiopaque substance from reservoir 45 into lumen 104 into the syringe and 2) by moving plunger 46 from position B to position A, the doctor can inject radiopaque substance into lumen 104 into catheter 19 into the vascular system of the patient by overcoming the force of spring 162 and approximate into position D. Once the doctor finishes injecting dye and taking x-rays of the patient's system and desired organ by simply arresting the syringe and the plunger, the patient's pressure in addition to force of spring 222 push valve 216 back to its resting position C counterclockwise and closing lumen 104 towards this range and putting lumen 104 in contact with pressure gauge 33. To repeat the x-ray pictures, only simple two steps can be performed by the doctor by moving plunger 46 from position A to position B and back to position A sucking out the dye from reservoir 45 and injecting it into lumen 104 and catheter 19 into the patient's vascular system. As the contrast dye enters the patient's vascular system, the doctor typically has a three to five second time in which to take x-rays as dye flows throw the artery or organ of interest. The doctor typically takes between two to five x-rays for each artery or organ of interest, and for each x-ray only two steps are required as I mentioned before. No need to move any exterior shutters or stopcocks in this improved system. The injection procedure of apparatus 200 is improved over that of conventional fluid delivery apparatus 10. In addition, unlike conventional fluid delivery apparatus 10, fluid delivery apparatus 200 provides the advantage of generally constant automatic blood pressure without any need to repeatedly move external controls. As noted previously, some organs require multiple catheters to get a complete x-ray of the organ and each time a new catheter must be utilized, the above described initialization and air clearing procedures must also be repeated for apparatus 200. The initialization procedure for apparatus 200 requires only three steps, only two of which require the movement of external controls. Therefore the initialization procedure of apparatus 200 is also improved over that of conventional apparatus 10.

Finally, when using either apparatus 100 or 200 in angiography a doctor may need to periodically flush catheter 19 to prevent blood from sitting motionless in catheter 19 thereby reducing the risk of clot formation. Only three steps are required to flush catheter 19 in either apparatus 10 or 200. First the doctor moves shutter 152 to its second position placing saline reservoir 37 in fluid communication with port 150 but closing radiopaque substance reservoir 45. At this point, saline does not flow from port 150 to lumen 104 because the flow of setting does not overcome the force of spring 162. Second, the doctor moves plunger 46 of syringe 20 from position A to position B. This plunger movement creates a vacuum in lumen 104 in the area generally below valve 158. This vacuum allows saline in port 150 to overcome the force of spring 162 automatically moving valve 162 to position H and drawing saline from port 150 into lumen 104. When the pressure in port 150 and lumen 104 equalize, the spring 162 moves valve 158 back to position G. Third, the doctor then has to move either handle 118 or string 218 to open lumen 104 and to close port 33. The doctor then moves plunger 46 all the way to position B while tapping on the system to clear it from any air bubbles that may be adhering to catheter 19 or lumen 104 paralyzing string 218 or moving handle 118 back to its vertical position so that the doctor can inject mixed blood saline from the syringe into lumen 104 and to catheter 19 flushing it from any blood and injecting saline into the catheter. That would initialize the system and make it ready and prevent any clotting and keep the system ready for more injection or for further operations. From the above, one skilled in the art will appreciate that apparatus 100 and 200 both facilitate a significantly improved flushing procedure from that of conventional apparatus 10. The flushing procedure of apparatus 10 requires six to seven steps, almost all of them require turning of the handles and stopcocks. In contrast, the flushing procedure of apparatus 100 and 200 requires only three steps, only two of them require movement of exterior shutters. The present invention is illustrated here in by example and various modifications may be made by a person of ordinary skill in the art. For example, numerous geometries and/or relative dimensions could be altered to accommodate specific applications of the improved fluid delivery apparatus. As another example, although the operation of the improved fluid delivery apparatus is described above in connection with angiography, the apparatus is operable with a variety of medical catheterization procedures. As a further example, the improved fluid delivery apparatus may be used with a pressurized radiopaque substance reservoir similar to the pressurized saline reservoir if desired. It is thus believed that the operation and construction of the present invention will be apparent from the foregoing description. While the method and apparatus shown or described has been characterized as being preferred, it would be obvious that various changes and modifications may be made therein without departing from the spirit and scope of the invention as defined in the following claims.

What is claimed is:

1. Fluid delivery apparatus, comprising:
a manifold having a lumen and a first port connected to said lumen, said first port having a first branch for connecting to a first fluid reservoir and a second branch for connecting to a radiopaque substance;
a shutter having first and second transverse bores and slidably disposed within said first and second branches;
wherein said shutter is moveable to a first position in which said first transverse bore places it first fluid reservoir and fluid communication with said first port and:
wherein said shutter is moveable to a second position in which said second transverse bore places said second fluid reservoir or a radiopaque substance in fluid communication with said first port.

2. The fluid delivery apparatus of claim 1 wherein:
said lumen has a first end for connecting with the catheter; and
said lumen has a second end for connecting with a syringe.

3. The fluid delivery apparatus of claim 2 further comprising:
a first valve rotatably coupled to an anterior surface of a said lumen proximate said first port.
a spring coupled to said first valve biasing said first valve against rotation in a direction towards said catheter.

4. The fluid delivery apparatus of claim 3 wherein said first valve has a resting position allowing fluid flow through said lumen but not into said first port.

5. The fluid delivery apparatus of claim 4 wherein said manifold further comprises a second port between the catheter disposed between said catheter and said first port and having a first end connected to said lumen.

6. The fluid delivery apparatus of claim 5 further comprising:
a second valve rotatably coupled to an anterior surface of said lumen, proximate said second port, and
a spring coupled to said second valve biasing said second valve against rotation in the direction towards said catheter.

7. The fluid delivery apparatus of claim 6 wherein the second valve has a resting position allowing fluid flow into said second port but not through said lumen.

8. The fluid delivery apparatus of claim 7 wherein said first fluid reservoir contains saline, said second fluid reservoir contains a radiopaque substance and said second port has a second end for connecting to pressure measuring means.

9. The fluid delivery apparatus of claim 3 wherein said first valve is rotatably coupled to an upper anterior surface of said lumen on a side of said first port closest to said catheter.

10. The fluid delivery apparatus of claim 9 wherein said first valve is rotatably coupled to said upper anterior surface of said lumen with a hinge.

11. The fluid delivery apparatus of claim 6 wherein said second valve is rotatably coupled to an upper anterior surface of said lumen on a side of said second port farthest from said catheter.

12. The fluid delivery apparatus of claim 11 wherein said second valve is rotatably coupled to said upper anterior surface of said lumen with a hinge.

13. The fluid delivery apparatus for delivery fluid to the vascular system of a patient, comprising:
a manifold having a lumen with a first end for connecting to a catheter and a second end for connecting to a syringe and a first port proximate said catheter having a first end connected to said lumen a second end for connecting to a pressure measuring means;
a first valve rotatably coupled to an anterior surface of said lumen proximate said first port and
a spring coupled to said first valve biasing said first valve against rotation in a direction towards said catheter.

14. The fluid delivery apparatus of claim 13 wherein said first valve has a resting position allowing fluid flow into said first port but not to said lumen.

15. The fluid delivery apparatus of claim 14 further comprising a hinge that rotatably couples said first valve to an upper anterior surface of said lumen on a side of said first port farthest from said catheter.

16. The fluid delivery apparatus of claim 15 further comprising an exterior handle rotatably couple d to said hinge.

17. The fluid delivery apparatus of claim 16 wherein said exterior handle is moveable to a first position placing said first valve in said resting position and to a second position where said first valve allows fluid flow to said lumen but not into said first port.

18. The fluid delivery apparatus of claim 17 wherein said manifold comprises the second port having a first end connected to said lumen and a second end for connecting to a fluid reservoir.

19. The fluid delivery apparatus of claim 15 wherein said manifold has a second port approximate said first port having a first end connected to said lumen and a second end open to an exterior of said manifold and further comprising:
an annular seal disposed in said second end of said second port and
a string coupled to said first valve and passing through an aperture of said annular seal.

20. The fluid delivery apparatus of claim 196 wherein said string may be moved to place said first valve in a second position allowing fluid to flow through said lumen but not into said first port.

21. The fluid delivery apparatus of claim 20 wherein said manifold comprises a third port having a first end connected to said lumen and a second end for connecting to a fluid reservoir.
